(19) United States
(12) Patent Application Publication
(10) Pub. No.: US 2007/0088404 A1
(43) Pub. Date: Apr. 19, 2007

(54) Methods and systems for improving neural functioning, including cognitive functioning and neglect disorders

(76) Inventors: Allen Wyler, Seattle, WA (US); Bradford Evan Gliner, Sammamish, WA (US)

Correspondence Address: PERKINS COIE LLP;PATENT-SEA, P.O. BOX 1247, SEATTLE, WA, 98111-1247, US

(21) Appl. No.: 11/254,060

(22) Filed: Oct. 19, 2005

(51) Int. Cl. A61N 1/39

(52) U.S. Cl. 607/46; 607/1

(57) ABSTRACT

Methods and systems for improving neural functioning, including cognitive functioning and neglect disorders, are disclosed. A method for treating a patient in accordance with one embodiment of the invention includes applying electrical stimulation beneath a patient's skull to improve neuropsychological functioning of the patient, and, after applying the electrical stimulation, evaluating the functioning of the patient. The method can further include a process based at least in part on results of the evaluation, with the process including maintaining and/or changing at least one parameter in accordance with which the electrical stimulation is applied, and/or ceasing to apply the electrical stimulation. Accordingly, aspects of the foregoing methods can be used to improve functioning in normal patients and/or patients suffering from disorders such as cognitive disorders.

### TECHNICAL FIELD

The present invention is directed generally toward methods and systems for improving neural functioning, including cognitive functioning. In particular embodiments, the methods and systems can be used to address neglect disorders.

### BACKGROUND

A wide variety of mental and physical processes are known to be controlled or influenced by neural activity in particular regions of the brain. In some areas of the brain, such as in the sensory or motor cortices, the organization of the brain resembles a map of the human body; this is referred to as the “somatotopic organization of the brain.” There are several other areas of the brain that appear to have distinct functions that are located in specific regions of the brain in most individuals. For example, areas of the occipital lobes relate to vision, regions of the left inferior frontal lobes relate to language in the majority of people, and regions of the cerebral cortex appear to be consistently involved with conscious awareness, memory, and intellect. This type of location-specific functional organization of the brain, in which discrete locations of the brain are statistically likely to control particular mental or physical functions in normal individuals, is herein referred to as the “functional organization of the brain.”

Many problems or abnormalities with body functions can be caused by damage, disease and/or disorders of the brain. A stroke, for example, is one very common condition that damages the brain. Strokes are generally caused by emboli (e.g., obstruction of a vessel), hemorrhages (e.g., rupture of a vessel), or thrombi (e.g., clotting) in the vascular system of a specific region of the cortex, which in turn generally causes a loss or impairment of a neural function (e.g., neural functions related to face muscles, limbs, speech, etc.). Stroke patients are typically treated using physical therapy to rehabilitate the loss of function of a limb or another affected body part. For most patients, little can be done to improve the function of the affected limb beyond the recovery that occurs naturally without intervention.

One existing physical therapy technique for treating stroke patients constrains or restrains the use of a working body part of the patient to force the patient to use the affected body part. For example, the loss of use of a limb is treated by restraining the other limb. Although this type of physical therapy has shown some experimental efficacy, it is expensive, time-consuming and little-used. Stroke patients can also be treated using physical therapy plus adjunctive therapies. For example, some types of drugs, including amphetamines, increase the activation of neurons in general. These drugs also appear to enhance neural networks. However, these drugs may have limited efficacy because their mechanisms of action are very non-selective and they cannot be delivered in high concentrations directly at the site where they are needed. Still another approach is to apply electrical stimulation to the brain to promote the recovery of functionality lost as a result of a stroke. While this approach has been generally effective, it has not adequately addressed all stroke symptoms.

One common syndrome following a stroke is neglect. Neglect is a cognitive defect that causes patients to lose cognizance of portions of their surroundings and/or themselves. Most frequently, neglect results from damage to the right (i.e., non-language) hemisphere of the brain, and affects the contralesional side of the patient and/or the patient's perception of his or her contralesional surroundings. For example, patients demonstrating neglect may fail to be aware of objects (including their own body parts) or people in the left half of the space around them. Patients suffering from neglect may fail to spontaneously move their eyes to the left, even though such movements are possible for the patient during formal testing. Patients may examine only half of a page presented before them, may be unable to bisect a line at its middle, may copy only half of a drawing positioned before them, may fail to groom the left side of their faces or heads, and/or may exhibit other such symptoms.

In many cases, the patient may be unaware of the fact that he or she exhibits the foregoing symptoms (i.e., if they are unaware of their paretic left arm they may deny any problem). Accordingly, treating neglect is often difficult because the patient is not motivated by the physically manifested reminders of the condition, though such reminders would appear to be continual and obvious to an observer. Therefore, there is a need to develop more effective and efficient treatments for rehabilitating stroke patients and patients that have other types of brain damage and/or can otherwise benefit from an improvement in cognitive functioning.

### BRIEF DESCRIPTION OF THE DRAWINGS

FIG. 1 is a flow diagram illustrating a process for improving neuropsychological functioning of a patient in accordance with an embodiment of the invention.

FIG. 2 is a left-side view of a patient's brain, identifying potential stimulation sites in accordance with embodiments of the invention.

FIG. 3 is a top view of a patient's brain illustrating further potential target stimulation sites in accordance with embodiments of the invention.

FIG. 4 is a partially schematic, isometric illustration of a magnetic resonance chamber in which a patient may be evaluated in accordance with an embodiment of the invention.

FIG. 5 illustrates a patient wearing a peripheral stimulation device that may be used in combination with evaluation devices in accordance with further embodiments of the invention.

FIG. 6 illustrates a patient wearing a network of electrodes positioned to detect brain activity in accordance with further embodiments of the invention.

FIG. 7 illustrates an electrical stimulation device implanted in a patient in accordance with an embodiment of the invention.

FIG. 8 illustrates an electrical device operatively coupled to an external controller in accordance with another embodiment of the invention.

FIG. 9 is a schematic illustration of a pulse system configured in accordance with an embodiment of the invention.

FIG. 10 is an isometric illustration of a device that carries electrodes in accordance with another embodiment of the invention.

FIG. 11 is a partially schematic, side elevation view of an electrode configured to deliver electromagnetic stimulation to a subcortical region in accordance with an embodiment of the invention.

### DETAILED DESCRIPTION

A. Introduction

The present invention is directed generally toward methods and systems for improving neural functioning, including cognitive functioning. A method in a particular aspect of the invention is directed to treating a patient by applying electrical stimulation beneath the patient's skull to improve neuropsychological functioning of the patient. After applying the electrical stimulation, the process can further include evaluating the functioning of the patient. Based at least in part on the results of the evaluation, the method can still further include changing and/or maintaining at least one parameter in accordance with which the electrical stimulation is applied, and/or ceasing to apply the electrical stimulation.

In further particular embodiments, the method can include selecting at least one type of cognitive functioning and, based at least in part on the selected type of cognitive functioning, selecting a target neural population to which the electrical stimulation is directed. The electrical stimulation can be applied at or beneath the patient's cortex and in at least some embodiments, can be applied to the parietal lobe of the brain. Electrical stimulation can be provided to improve the patient's memory, effectuate a lasting change in the patient's cognitive functioning, and/or be applied to a patient having a perceptual disorder. In other embodiments, electrical stimulation can be provided to a patient having generally normal cognitive functioning. In still further embodiments, electrical stimulation can be provided to improve a neuropsychiatric functioning of the patient.

In yet another embodiment, a method for treating a patient having a neglect disorder can include applying electromagnetic stimulation to the patient's brain to at least partially reduce the effects of the neglect disorder. The method can further include determining a severity of the neglect disorder by administering a neglect test to the patient after applying the electromagnetic stimulation. Based at least in part on the results of the neglect test, the method can further include changing at least one parameter in accordance with which the electromagnetic stimulation is applied, or ceasing to apply the electromagnetic stimulation, or both.

B. Methods for Improving a Patient's Functioning

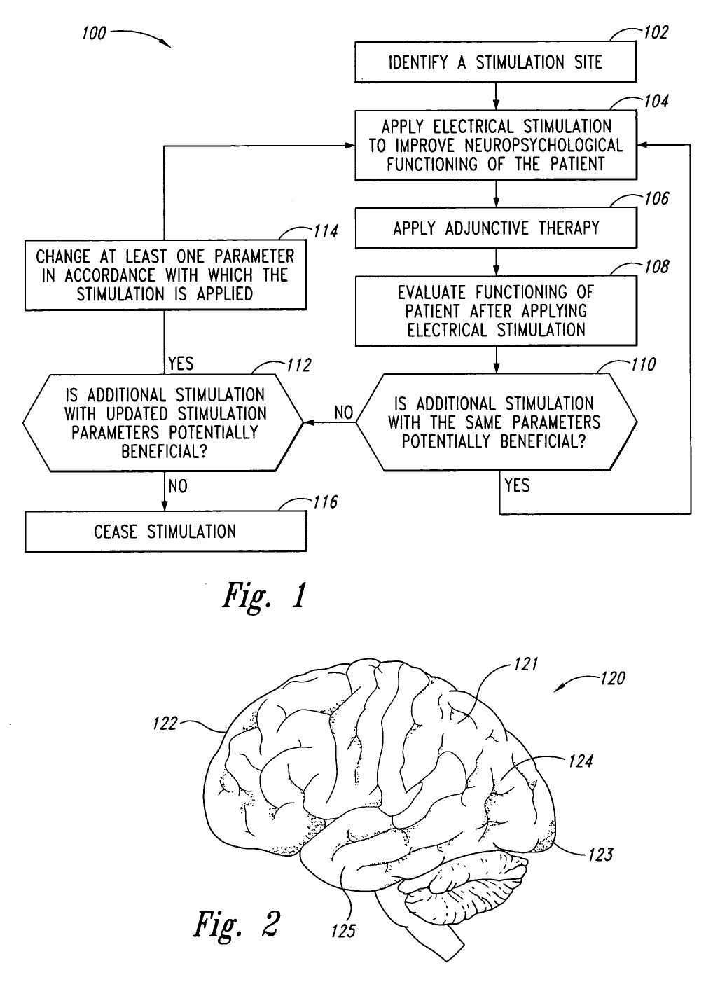

FIG. 1 is a flow diagram illustrating a method 100 for improving a patient's neuropsychological functioning in accordance with an embodiment of the invention. Further details regarding the processes identified in FIG. 1 are described below with reference to FIGS. 2-11. Beginning with FIG. 1, process portion 102 includes identifying a stimulation site. The stimulation site is typically located at the patient's central nervous system, and in many instances, is located at the patient's brain. In process portion 104, electrical stimulation is applied to the patient's central nervous system (e.g., beneath the patient's skull) to improve the neuropsychological functioning of the patient. In particular embodiments, the electrical stimulation can enhance the patient's naturally occurring efforts to recruit neural cells to take over functions performed by damaged cells (e.g., based on neuroplasticity). The electrical stimulation can be applied in association with an adjunctive therapy, as indicated by process portion 106. The adjunctive therapy can be selected based at least in part upon the particular symptoms the patient exhibits, so as to at least partially address those symptoms.

Process portion 108 can include evaluating the functioning of the patient after the electrical stimulation has been applied. Based at least in part on the results of the evaluation, process portion 110 can include determining whether additional stimulation with the same stimulation parameters is potentially beneficial. If so, then the process returns to process portion 104. If not, then in process portion 112, it can be determined whether additional stimulation with different parameters may be potentially beneficial. If so, then in process portion 114 at least one of the stimulation parameters can be changed, and the process can return to process portion 104 for application of additional electrical stimulation to the patient. If not, then in process portion 116, the electrical stimulation ceases.

C. Identifying a Stimulation Site

FIG. 2 is a side illustration of the brain 120 illustrating the four major brain lobes, e.g., the parietal lobe 121, the frontal lobe 122, the occipital lobe 124 (which includes the visual cortex 123), and the temporal lobe 125. In many cases, patients suffering from neglect may benefit from stimulation at the parietal lobe 121, and/or the frontal lobe 122. In other embodiments, cognitive functioning and/or neuropsychological functioning can be improved by stimulation at the occipital lobe 124 and/or the temporal lobe 125. Accordingly, the practitioner can select a stimulation site that is consistent with the patient's condition.

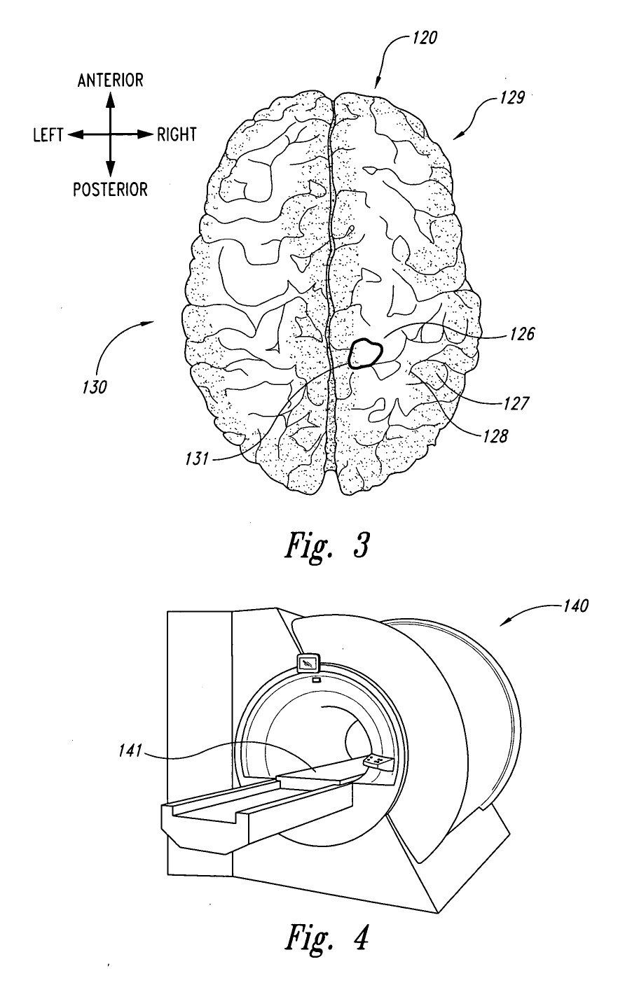

FIG. 3 is a top view of the brain 120 illustrating particular aspects of the parietal lobe 121, including the superior parietal lobule 126, the inferior parietal lobule 127, and the intraparietal sulcus 128. FIG. 3 also illustrates a target neural population 131 located at the superior parietal lobule 126 of the patient's right brain hemisphere 129. As described above, many patients suffering from neglect suffer from neglect of the left side of their bodies or fields of view, and accordingly, may benefit from the stimulation of the right hemisphere. In other embodiments, the patient's cognitive and/or other functioning may be improved by stimulating the left hemisphere 130. Depending upon embodiment details and/or the nature or extent of a patient's neurologic dysfunction, the patient may benefit from neural stimulation directed toward one or more target neural populations, which may reside in one or both brain hemispheres. Further details regarding the particular sites selected for stimulation are described below.

In particular embodiments, one or more target neural population 131 can be selected based on past experience with patients presenting with similar symptoms. For example, if over the course of time, it is determined that stimulating the superior parietal lobe 126 is particularly effective for treating one or more types of neglect, electrical stimulation can be applied at this location in patients exhibiting the corresponding symptom(s). In other embodiments, selecting a set of target neural populations 131 can be performed on a patient-specific (e.g., patient-by-patient) basis. For example, the particular portion of the brain that benefits from electrical stimulation may vary from patient to patient, even for patients presenting with similar or identical symptoms. In such cases, techniques can be used to identify the areas of the brain well suited for electrical stimulation for each individual patient. In many instances, this process can include (a) providing a stimulus that causes the patient to exhibit a problematic symptom, and then (b) simultaneously identifying areas of the brain that are either active, or are inactive, but should be active. Accordingly, identifying target stimulation areas can include (a) identifying lesioned or other damaged areas, (b) identifying areas adjacent or proximate to the damaged areas, and/or (c) identifying other areas expected to assume, at least in part, the functions of a damaged area, or otherwise improve the functionality of the patient. FIGS. 4-6 illustrate representative techniques for performing such identification tasks.

FIG. 4 illustrates a magnetic resonance system 140 having a patient platform 141 for carrying the patient while a practitioner identifies one or more electrical stimulation sites. If the stimulation site is to be located based on previous data for similarly situated patients, the magnetic resonance system 140 can be used to provide magnetic resonance imaging (MRI) data that are in turn used to locate target brain areas relative to patient-specific features (e.g., anatomical features or fiducials). In other embodiments, the system 140 can provide functional MRI (fMRI) results. For example, the patient can be placed in the system 140 and asked to perform a task that causes the patient to exhibit the problematic symptom. The data obtained while the patient is in system 140 can then be used to identify where active and/or inactive brain regions are located, which can in turn provide information for identifying the electrical stimulation sites. The data can be in the form of human-readable images, and/or computer-readable output.

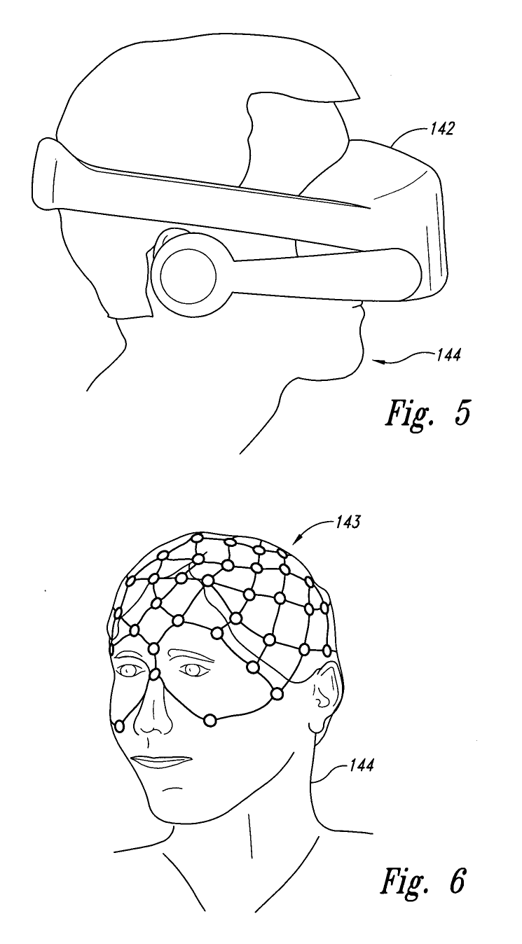

Because the system 140 tends to be loud and confined, it may be difficult to provide the peripheral stimulus and/or gauge the patient's response to the peripheral stimulus while the patient is in the system chamber. In some instances, the stimulus can include asking the patient a question (via a headset, speaker system or other peripheral stimulation device), and the patient can respond verbally via a microphone system. In other instances, for example, when the stimulus is of a more complex visual nature, the patient may be outfitted with another type of peripheral stimulation device. Referring now to FIG. 5, such a peripheral stimulation device 142 can include virtual reality goggles placed on the patient 144 before the patient is placed within the chamber 140. The patient can view a visually-based test (e.g., a bells cancellation test or a matrix reasoning test) via the peripheral stimulation device 142, and can provide a response by voice, or by pressing a hand-held key, moving a joystick, or by another suitable method (e.g., through a choice or selection made by an eye movement recognized by an ocular monitoring/tracking device incorporated into a headset or virtual reality goggles). The peripheral stimulation device 142 and any device used to transmit the patient's response can be compatible with the system 140. For example, these devices can be operated by fiber optic links and/or can otherwise be compatible with the strong magnetic fields associated with the system 140.

In other embodiments, other techniques, such as EEG techniques, can be used to identify the activity in the patient's brain while the patient responds to a stimulus. FIG. 6 illustrates the patient 144 wearing an electrode or sensor net 143 (e.g., a geodesic sensor net manufactured by Electrical Goedesics, Inc., of Eugene, Oregon) that includes a network of receptor electrodes positioned over the patient's scalp. When the patient is provided with an external or peripheral stimulus (e.g., a cognitive or other type of test) the patient's brain generates electrical signals in response to the stimulus, and these signals can be identified by the electrode net 143. The waveform, spatial, and/or temporal characteristics of the signals (or the absence of signals) can be used to identify one or more target neural populations. The patient's performance on the test (e.g., how accurately the patient answers particular questions, or identifies particular objects) can be separately tracked to identify the severity of the patient's symptoms and/or the pace of the patient's progress during the course of treatment.

In other embodiments, other techniques can be used to locate areas of the brain at which electrical stimulation may provide a benefit. Such techniques can include magnetic resonance spectroscopy (MRS) techniques (which can identify the presence and relative levels of particular neurochemical species) to identify neurotransmitter imbalances or states associated with neuropsychiatric and/or other disorders, PET techniques, optical tomography techniques, and/or other techniques. In any of these embodiments, various techniques can be used to identify areas (e.g., neuroplastic areas) that can take over functions for other brain areas, improve on an existing level of functioning, and/or otherwise provide a benefit to the patient, as a direct or indirect result of electrical stimulation.

D. Applying Electrical Stimulation

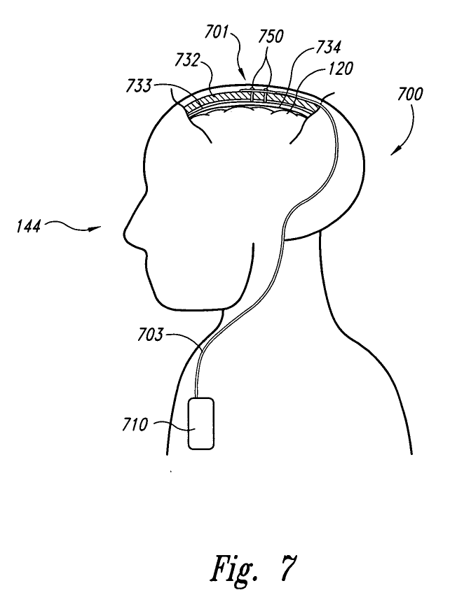

Once the electrical stimulation site or sites have been identified, an electrical stimulation device may be positioned at a location to provide electrical stimulation to the selected sites. FIGS. 7-11 illustrate representative devices for accomplishing this function. FIG. 7 is a schematic illustration of a neurostimulation system 700 implanted in the patient 144 to provide stimulation in accordance with several embodiments of the invention. The system 700 can include an electrode device 701 carrying one or more electrodes 750. The electrode device 701 can be positioned in the skull 732 of the patient 144, with the electrodes 750 positioned to stimulate target areas of the brain 120. For example, the electrodes 750 can be positioned just outside the dura mater 733 (which surrounds the brain 120) to stimulate cortical tissue. In another embodiment described later with reference to FIG. 11, an electrode can penetrate the dura mater 733 to stimulate subcortical tissues. In still further embodiments, the electrodes 750 can penetrate the dura mater 733 but not the underlying pia mater 734, and can accordingly provide stimulation signals through the pia mater 734.

The electrode device 701 can be coupled to a pulse system 710 with a communication link 703. The communication link 703 can include one or more leads, depending (for example) upon the number of electrodes 750 carried by the electrode device 701. The pulse system 710 can direct electrical signals to the electrode device 701 to stimulate target neural tissues.

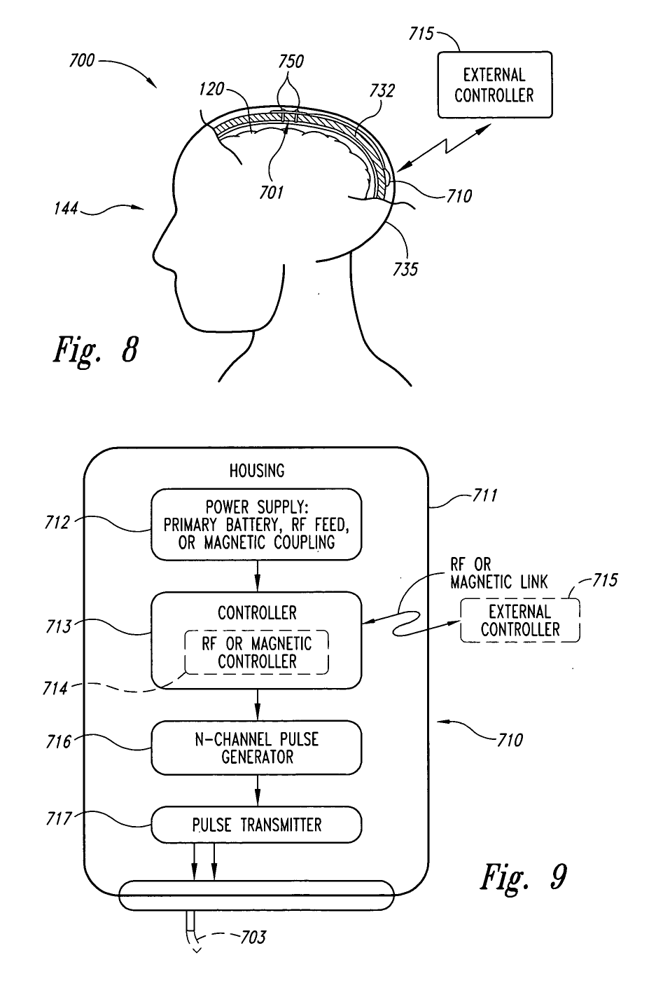

The pulse system 710 can be implanted at a subclavicular location, as shown in FIG. 7. In particular embodiments, the pulse system 710 (and/or other implanted components of the system 700) can include titanium and/or other materials that can be exposed to magnetic fields generated by magnetic resonance systems (e.g., the system shown in FIG. 4) without harming the patient. The pulse system 710 can also be controlled internally via pre-programmed instructions that allow the pulse system 710 to operate autonomously after implantation. In other embodiments, the pulse system 710 can be implanted at other locations, and at least some aspects of the pulse system 710 can be controlled externally. For example, FIG. 8 illustrates an embodiment of the system 700 in which the pulse system 710 is positioned on the external surface of the skull 732, beneath the scalp 735. The pulse system 710 can be controlled internally and/or via an external controller 715.

FIG. 9 schematically illustrates a representative example of a pulse system 710 suitable for use in the neural stimulation system 700 described above. The pulse system 710 generally includes a housing 711 carrying a power supply 712, an integrated controller 713, a pulse generator 716, and a pulse transmitter 717. The power supply 712 can be a primary battery, such as a rechargeable battery or other suitable device for storing electrical energy. In other embodiments, the power supply 712 can be an RF transducer or a magnetic transducer that receives broadcast energy emitted from an external power source and that converts the broadcast energy into power for the electrical components of the pulse system 710.

In one embodiment, the integrated controller 713 can include a processor, a memory, and a programmable computer medium. The integrated controller 713, for example, can be a microcomputer, and the programmable computer medium can include software loaded into the memory of the computer, and/or hardware that performs the requisite control functions. In another embodiment identified by dashed lines in FIG. 9, the integrated controller 713 can include an integrated RF or magnetic controller 714 that communicates with the external controller 715 via an RF or magnetic link. In such an embodiment, many of the functions performed by the integrated controller 713 may be resident on the external controller 715 and the integrated portion 714 of the integrated controller 713 may include a wireless communication system.

The integrated controller 713 is operatively coupled to, and provides control signals to, the pulse generator 716, which may include a plurality of channels that send appropriate electrical pulses to the pulse transmitter 717. The pulse generator 716 may have multiple channels, with at least one channel associated with a particular one of the electrodes 750 described above. The pulse generator 716 sends appropriate electrical pulses to the pulse transmitter 717, which is coupled to a plurality of the electrodes 750 (FIG. 1). In one embodiment, each of these electrodes 750 is configured to be physically connected to a separate lead, allowing each electrode 750 to communicate with the pulse generator 716 via a dedicated channel. Suitable components for the power supply 712, the integrated controller 713, the external controller 715, the pulse generator 716, and the pulse transmitter 717 are known to persons skilled in the art of implantable medical devices.

The pulse system 710 can be programmed and operated to adjust a wide variety of stimulation parameters, for example, which electrodes are active and inactive, whether electrical stimulation is provided in a unipolar or bipolar manner, and/or how the stimulation signals are varied. In particular embodiments, the pulse system 710 can be used to control the polarity, frequency, duty cycle, amplitude, and/or spatial and/or temporal qualities of the stimulation. The stimulation can be varied to match naturally occurring burst patterns (e.g., theta burst stimulation), and/or the stimulation can be varied in a predetermined, pseudorandom, and/or a periodic manner at one or more times and/or locations. Various systems and/or procedures for providing and/or varying neural stimulation in manners that may be relevant to particular embodiments of the invention are described in detail in U.S. application Ser. No. 11/182,713, entitled “Systems and Methods for Enhancing or Affecting Neural Stimulation, Efficiency and/or Efficacy, filed on Jul. 15, 2005, which is incorporated herein by reference in its entirety.

E. Adjunctive Therapies

A given treatment regimen may also include, in addition to electrical stimulation, one or more adjunctive or synergistic therapies to facilitate enhanced symptomatic relief and/or at least partial recovery from neurological dysfunctions. An adjunctive or synergistic therapy may include a behavioral therapy, such as a physical therapy activity, a movement and/or balance exercise, an activity of daily living (ADL), a vision exercise, a reading exercise, a speech task, a memory or concentration task, a visualization or imagination exercise, an auditory activity, an olfactory activity, a relaxation activity, and/or another type of behavior, task or activity. When a patient is being treated for neglect, the patient may undertake tasks that specifically engage a transition from a perceived region into a neglected region. For example, therapy may include applying stimulation while the patient tracks a light from a portion of the right extrapersonal space to the left extrapersonal space. In another embodiment, the patient may track a somatic simulation from right to left relative to his or her body, or drag a block from right to left to hit a target (e.g., on a display device). Further examples of representative adjunctive therapies are disclosed by Tripathi et al. in a paper entitled, “Rehabilitation of patients with hemispatial neglect using visual-haptic feedback in virtual reality environment,” (International Conference on Human-Computer Interaction HCII, 2005), incorporated herein by reference. In other embodiments, the adjunctive therapy can include the introduction of a drug or other chemical substance into the patient's body. The adjunctive therapy can be provided before, during and/or after the electrical stimulation during a given treatment session. When the adjunctive therapy is provided before or after the electrical stimulation, the temporal spacing between the electrical stimulation and the adjunctive therapy can be selected to provide a desired effect. In any of these embodiments, the relative timing between the electrical stimulation portion of the treatment regimen and the adjunctive therapy portion of the treatment regimen can be controlled and/or altered during the course of the treatment regimen.

The particular adjunctive therapy selected can depend upon the symptoms the particular patient exhibits. For example, if the patient exhibits spatial neglect, the selected adjunctive therapy may be different than if the patient exhibits another cognitive defect (e.g., memory loss). In some instances, the adjunctive therapy can be similar or at least partially similar to an evaluation technique that may be performed to gauge the severity level of the patient's dysfunction. For example, if a patient performs a bells cancellation test as an evaluation technique for determining the severity of a spatial neglect dysfunction, the patient may engage in a similar or identical exercise as part of an adjunctive therapy. In any of these embodiments, it is believed that the adjunctive therapy can improve on and/or make more permanent the results obtained from applying electrical stimulation alone.

In another particular example, a patient suffering from neglect can have electrical stimulation applied at the cortex (e.g., at the right parietal lobe) and possibly other central nervous system locations (a) while stimulating the neglected parts of the body, or (b) while the patient tries to use or move those body parts, and/or (c) while a practitioner passively moves those body parts. The cortical stimulation can be performed independently of, simultaneously with, or in a temporally sequenced manner (e.g., based upon an estimated or measured neural signaling latency) with sensory stimulation and/or peripheral stimulation (e.g., Functional Electrical Stimulation (FES)) to strengthen neural signaling input to healthy or surviving brain tissue. For visual neglect, the cortical stimulation can be applied to surviving areas around and/or associated with (e.g., having neural projections into) the occipital visual cortex, while the sensory stimulation can be provided visually.

F. Evaluating the Functioning of the Patient

At periodic intervals during the course of a treatment regimen, the patient's level of functioning can be evaluated. In some instances, for example, if the adjunctive therapy applied to the patient includes an evaluative test, the evaluation can be conducted during each therapy session by tracking patient performance on the test. In other embodiments, the evaluation can be provided on a less frequent basis and/or via other techniques.

As described above, one method for performing an evaluation is to administer a symptom-specific type of test. Such a test can include a bells cancellation test for neglect or other tests for other specific symptoms, including other cognitive deficits such as memory deficits. In many of these tests, the evaluation includes, and is based at least in part on, an active motor response by the patient. For example, if the patient is instructed to draw an object, identify objects, or respond verbally to a query, the response includes a motor response as well as a cognitive response. The nature of the test can be focused on the cognitive response and, to the extent the patient has motor deficits in addition to cognitive deficits, the test results can be segregated into cognitive-based results and motor-based results so that each can be tracked independently.

In other embodiments, the patient's functioning can be evaluated by evaluating a physiologic function that corresponds to a neuropsychological functioning level of the patient. Such an evaluation can be based on changes in neurotransmitter levels (e.g., using MRS), or changes in cerebral blood flow or other parameters that correlate with neural functioning. For example, a cognitively dysfunctional patient may exhibit a relatively small change in cerebral blood flow (at the appropriate brain location) when engaging in a cognitive task, while a more fully functioning patient may exhibit a larger change in cerebral blood flow. Accordingly, identifying a difference between cerebral blood flow at one or more times, or the difference between a small change in cerebral blood flow and a large change in cerebral blood flow can indicate an improvement in cognitive functioning. In other embodiments, other physiological changes (e.g., changes in neuronal signals) or differences in changes can provide similar information. Further details regarding such techniques are described in the following copending patent applications, filed concurrently herewith, and incorporated herein by reference: U.S. application Ser. No. ______, titled “Methods and Systems for Establishing Parameters for Neural Stimulation” (Attorney Docket No. 33734.8079US) and U.S. application Ser. No. ______, titled “Neural Stimulation and Optical Monitoring Systems and Method” (Attorney Docket No. 33734.8084US).

The type of evaluation technique selected for a given patient may depend at least in part on the nature of the electrical stimulation device implanted in the patient. For example, in some cases, magnetic resonance techniques such as fMRI can be used to identify and/or evaluate neural changes associated with the patient's level of functioning. If the patient is to undergo evaluation while in a magnetic resonance chamber, the practitioner first establishes that the electrical stimulation device is compatible with such techniques, and does not create unwanted electromagnetic or thermal effects in the patient's brain. If it is expected that the patient will undergo exposure to strong magnetic fields, the practitioner may elect to implant stimulation devices (e.g., a magnetic resonance compatible IPG, and/or one or more microstimulators such as BIONS™ (Advanced Bionics Corporation, Sylmar, Calif.)) that are compatible with magnetic fields found in magnetic resonance environments. In other embodiments, other techniques can be used to evaluate the patient's functionality level without subjecting the patient to strong magnetic fields. For example, functional optical imaging, and/or EEG using an electrode or sensor net similar to that described above with reference to FIG. 6 can be used to evaluate the patient's improvement, neurofunctional condition or change, or performance.

G. Changing Test Parameters at Least in Part on the Basis of the Evaluation

In some instances, the results of the foregoing evaluation can have a direct or indirect effect on the selection of parameters for electrically stimulating the patient. For example, if the evaluation indicates that the patient's performance is improving at an expected rate, the stimulation parameters need not be changed. If the evaluation indicates that the patient's progress has leveled off, one or more stimulation parameters may be changed to further increase patient functioning. If, after multiple parameter changes, no further change in patient functioning results, the electrical stimulation program can be interrupted for a given time period (e.g., a number of weeks over which neural consolidation may occur), or halted.

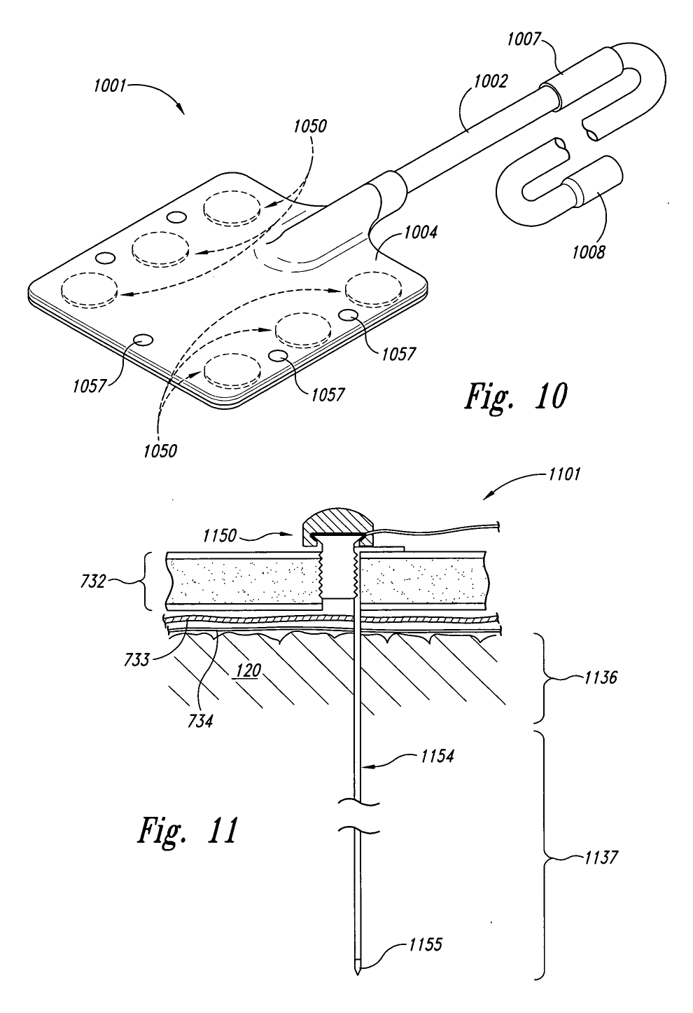

Any of a wide variety of stimulation parameters can be changed to expand upon and/or solidify the functional gains experienced by the patient. Such parameters can include the polarity of the electrical stimulation (e.g., anodal or cathodal), the manner in which the stimulation is applied (e.g., bipolar or monopolar), the location of the stimulation, and/or the waveform of the stimulation. For example, the current, voltage, frequency, pulse width, interpulse interval and/or other waveform-related functions can be changed to improve patient gains. Representative ranges for these parameters include: pulse widths from 50-300 μ/sec, frequencies from 1-200 Hz, current from 2-10 mA, voltage from 2-15V and interpulse intervals from 1-1000 msec. Also, to reduce any effect that neural adaptation and/or habituation may have on clinical benefit, random variations in parameters may be programmed into the pulse delivery system. The location at which the stimulation signals are provided may be changed by activating different electrodes on a particular electrode device, (e.g., using a device generally similar to the one described below with reference to FIG. 10). In other embodiments, additional electrode devices may be implanted within the patient's skull to effectively change the stimulation location.

H. Electronic Devices in Accordance with Further Embodiments

Stimulation can be provided to the patient using devices in addition to or in lieu of those described above. For example, FIG. 10 is a top, partially hidden isometric view of an embodiment of an electrode device 1001 configured to carry multiple cortical electrodes 1050. The electrodes 1050 can be carried by a flexible support member 1004 (located within the patient's skull) to place each electrode 1050 at a stimulation site of the patient when the support member 1004 is implanted within the patient's skull. Electrical signals can be transmitted to the electrodes 1050 via leads carried in a communication link 1003. The communication link 1003 can include a cable 1002 that is connected to the pulse system 710 (FIG. 7) via a connector 1008, and is protected with a protective sleeve 1007. Coupling apertures or holes 1057 can facilitate temporary attachment of the electrode device 1001 to the dura mater at, or at least proximate to, a stimulation site. The electrodes 1050 can be biased cathodally and/or anodally, as described above. In an embodiment shown in FIG. 10, the electrode device 1001 can include six electrodes 1050 arranged in a 2×3 electrode array (i.e., two rows of three electrodes each), and in other embodiments, the electrode device 1001 can include more or fewer electrodes 1050 arranged in symmetrical or asymmetrical arrays. The particular arrangement of electrodes 1050 can be selected based on the region of the patient's brain that is to be stimulated, and/or the patient's condition.

FIG. 11 illustrates an electrode device 1101 that may be configured to apply electrical stimulation signals to a cortical region 1136 or a subcortical region 1137 of the brain 120 in accordance with further embodiments of the invention. The electrode device 1101 can include an electrode 1150 having a head and a threaded shaft that extends through a pilot hole in the patient's skull 732. If the electrode 1150 is intended for cortical stimulation, it can extend through the skull 732 to contact the dura mater 733 or the pia mater 734. If the electrode 1050 is to be used for subcortical stimulation, it can include an elongate conductive member 1154 that extends downwardly through the cortical region 1136 into the subcortical region 1137. Most of the length of the elongate conductive member 1154 can be insulated, with just a tip 1155 exposed to provide electrical stimulation in only the subcortical region 1137. Subcortical stimulation may be appropriate in at least in some instances, for example, when the brain structures such as the basal ganglia are to be stimulated. In other embodiments, other deep brain structures (e.g., the amygdala or the hippocampus) can be stimulated using a subcortical electrode. If the hippocampus is to be stimulated, stimulation may be provided to the perihippocampal cortex using a subdurally implanted electrode that need not penetrate through brain structures other than the dura.

Further details of electrode devices that may be suitable for electromagnetic stimulation in accordance with other embodiments of the invention are described in the following pending U.S. Patent Applications, all of which are incorporated herein by reference: Ser. No. 10/891,834, filed Jul. 15, 2004; Ser. No. 10/418,796, filed Apr. 18, 2003; and Ser. No. 09/802,898, filed Mar. 8, 2001. Further devices and related methods for providing neural stimulation and adjunctive therapy are described in a copending U.S. application Ser. No. ______, titled “Systems and Methods for Patient Interactive Neural Stimulation and/or Chemical Substance Delivery,” (Attorney Docket No. 33734.8082US) filed concurrently herewith and incorporated herein by reference.

In still further embodiments, other techniques may be used to provide stimulation to the patient's brain. Such techniques can include electromagnetic techniques in addition to purely electrical techniques. In particular, such techniques can include transcranial magnetic stimulation techniques, which do not require that an electrode be implanted beneath the patient's skull. In still further embodiments, other techniques, which also may not require an implant, can be used. Such additional techniques can include transcranial direct current stimulation.

One feature of several embodiments of the methods and devices described above is that they can be used to improve the neuropsychological functioning of a patient. For example, by selecting a set of stimulation site based on historic data and/or the characteristics of a specific patient, and then providing electrical stimulation at one or more stimulation sites, possibly in association or conjunction with one or more adjunctive therapies (in which a type of adjunctive therapy selected may correspond to a stimulation site under consideration), a long-lasting change in the patient's neuropsychological functioning can be achieved. The long-lasting change can last for many weeks, months, or years, while the application of the treatment may be provided over a significantly shorter period of time (e.g., over a single period or temporally separated periods of about three weeks, about six weeks, or about two to eight weeks).

Another feature of embodiments of the methods and devices described above is that they can include updating the parameters with which stimulation is applied to the patient, based on an evaluation of the patient. The evaluation can include a test (e.g., a cognitive test), or another suitable evaluation of the patient's level of functioning. Accordingly, a given therapy program can be changed dynamically to account for individual patient performance.

The foregoing techniques can be applied to patients having a wide variety of neuropsychological dysfunctions. Such dysfunctions include neglect dysfunctions, which can in turn include unilateral neglect (e.g., sensory neglect, motor neglect, representational neglect, personal neglect, or spatial neglect). In other embodiments, other perceptual and/or cognitive disorders can be treated using these techniques. Such disorders can relate to the patient's vision (e.g., visual field cut, cortical blindness, or central achromatopsia), or loss of tactile and/or other sensations (hemianesthesia, Balint's syndrome, sensory extinction, and others). These disorders may arise in connection with a stroke, other brain lesion, or other brain trauma.

Any of the foregoing techniques can be used to treat a patient having a neurological dysfunction (e.g., a neglect dysfunction, and/or another cognitive dysfunction). In other embodiments, the foregoing techniques can be applied to patients functioning at normal levels or above normal levels to further improve patient functioning. In still further embodiments, techniques generally similar to the foregoing techniques can be used to address neuropsychiatric disorders, including but not limited to depression or post-traumatic stress disorder. In these embodiments, the methods used to identify stimulation sites and track patient progress may be selected to focus on neuropsychiatric indicators. Such methods can include identifying cortical areas, subcortical areas, and/or associated neural projections that may exhibit and/or influence (e.g., as a result of neural stimulation) neurotransmitter levels which, as described above, can be identified using MRS techniques.

In still further embodiments, techniques generally similar to those described above can be used to treat other disorders or functional deficits. For example, such techniques can be used to treat learning disabilities and/or dyslexia. In other instances, the disorders described above may result from conditions other than those described above. For example, while neglect is often associated with stroke patients, it may also result from plaque formations (associated with Alzheimer's disease) or neurodepletion (associated with Parkinson's disease).

From the foregoing, it will be appreciated that specific embodiments of the invention have been described herein for purposes of illustration, but that various modifications may be made without deviating from the invention. For example, certain aspects of the methods described above may be automated or partially automated, and may be implemented on computer systems and/or via computer-readable media. In particular embodiments, aspects of the stimulation site selection procedure and/or the evaluation procedure can be automated in such a fashion. Aspects of the invention described in the context of particular embodiments may be combined or eliminated in other embodiments. For example, in some cases a treatment regimen can proceed without an adjunctive therapy. Although advantages associated with certain embodiments of the invention have been described in the context of those embodiments, other embodiments may also exhibit such advantages. Additionally, none of the foregoing embodiments need necessarily exhibit such advantages to fall within the scope of the invention. Accordingly, the invention is not limited except as by the appended claims.

I/We claim:

1. A method for treating a patient, comprising:
applying electrical stimulation beneath a patient's skull to improve neuropsychological functioning of the patient;
evaluating the functioning of the patient; and
based at least in part on results of the evaluation, performing at least one of the following functions:
(a) changing at least one parameter in accordance with which the electrical stimulation is applied;
(b) ceasing to apply the electrical stimulation; and
(c) maintaining stimulation parameters in accordance with which the electrical stimulation is applied.

2. The method of claim 1, further comprising selecting a target neural population to which the electrical stimulation is directed.

3. The method of claim 1, further comprising implanting an electrical stimulation device beneath the patient's skull.

4. The method of claim 3, further comprising performing an evaluation of the patient while the electrical stimulation device is implanted by exposing the patient to magnetic fields.

5. The method of claim 1 wherein evaluating the functioning of the patient includes evaluating the functioning of the patient using magnetic resonance techniques.

6. The method of claim 1 wherein evaluating the functioning of the patient includes evaluating a physiologic function that corresponds to neuropsychological functioning of the patient.

7. The method of claim 1 wherein evaluating the functioning of the patient includes administering a test to the patient.

8. The method of claim 7 wherein administering a test to the patient includes administering a test that elicits a cognitive response on the part of the patient.

9. The method of claim 1 wherein improving the functioning of the patient includes improving cognitive functioning of the patient

10. The method of claim 1, further comprising:
selecting at least one type of cognitive functioning; and
based at least in part on the selected type of cognitive functioning, selecting a target neural population to which the electrical stimulation is directed.

11. The method of claim 1, further comprising:
selecting at least one type of cognitive functioning; and
based at least in part on the selected type of cognitive functioning, selecting a cognitive test, and wherein evaluating the functioning includes administering the cognitive test.

12. The method of claim 1 wherein evaluating the functioning of the patient includes administering a cognitive test that results in a cognitive response and a motor response by the patient, and wherein the method further comprises distinguishing the cognitive response from the motor response.

13. The method of claim 1 wherein applying electrical stimulation to the patient includes applying electrical stimulation to the cortex of the patient's brain.

14. The method of claim 1 wherein applying electrical stimulation includes applying electrical stimulation beneath the patient's cortex.

15. The method of claim 1 wherein applying electrical stimulation includes applying electrical stimulation to the parietal lobe.

16. The method of claim 1 wherein applying electrical stimulation includes applying electrical stimulation to improve the patient's verbal conceptualization.

17. The method of claim 1 wherein applying electrical stimulation includes applying electrical stimulation to improve the patient's memory.

18. The method of claim 1 wherein applying electrical stimulation includes applying electrical stimulation to effectuate a lasting change in the patient's cognitive functioning.

19. The method of claim 1 wherein applying electrical stimulation includes applying electrical stimulation to a patient having generally normal cognitive functioning.

20. The method of claim 1 wherein applying electrical stimulation includes applying electrical stimulation to a post-stroke patient.

21. The method of claim 1 wherein applying electrical stimulation includes applying electrical stimulation to a patient having a perceptual disorder.

22. The method of claim 1 wherein applying electrical stimulation includes applying electrical stimulation to a patient having a cognitive defect.

23. The method of claim 1 wherein applying electrical stimulation includes applying electrical stimulation to a patient having a neglect disorder.

24. The method of claim 23 wherein applying electrical stimulation includes applying electrical stimulation to the parietal lobe, and wherein the method further comprises selecting a portion of the parietal lobe to which stimulation is applied based at least in part on the type of neglect the patient suffers from.

25. The method of claim 1 wherein changing at least one parameter in accordance with which the electrical stimulation is applied includes changing at least one of a current, voltage and waveform of a stimulation signal applied to the patient.

26. The method of claim 1 wherein changing at least one parameter in accordance with which the electrical stimulation is applied includes changing a location at which the stimulation is applied.

27. The method of claim 1, further comprising directing the patient to engage in an adjunctive therapy.

28. The method of claim 27 wherein the adjunctive therapy is selected to include a cognitive task.

29. The method of claim 27 wherein the adjunctive therapy is selected to include a motor task.

30. The method of claim 27 wherein the adjunctive therapy is selected to include behavioral therapy.

31. The method of claim 37 wherein directing the patient to engage in an adjunctive therapy includes directing the patient to engage in an adjunctive therapy during a treatment session that also includes applying the electromagnetic stimulation.

32. A method for treating a patient, comprising:
applying electrical stimulation beneath a patient's skull to improve neuropsychiatric functioning of the patient;
evaluating the functioning of the patient; and
based at least in part on results of the evaluation, performing at least one of the following functions:
(a) changing at least one parameter in accordance with which the electrical stimulation is applied;
(b) ceasing to apply the electrical stimulation; and
(c) maintaining stimulation parameters in accordance with which the electrical stimulation is applied.

33. The method of claim 32, further comprising selecting a target neural population to which the stimulation is directed.

34. The method of claim 33 wherein the patient has a depression condition, and wherein the selecting a target neural population includes selecting a target neural population based at least in part on neurotransmitter imbalances.

35. The method of claim 33 wherein the patient has a depression condition, and wherein the selecting a target neural population includes selecting a target neural population based at least in part on neurotransmitter imbalances detected using a magnetic resonance spectroscopy technique.

36. The method of claim 32 wherein the patient has a depression condition, and wherein the evaluation includes an evaluation of the patient's depression condition.

37. A method for treating a patient having a neglect disorder, comprising:
applying electromagnetic stimulation to the patient's brain to at least partially reduce effects of the neglect disorder;
determining a severity of the neglect disorder by administering a neglect test to the patient after applying the electromagnetic stimulation; and
based at least in part on results of the neglect test, performing at least one of the following functions:
(a) changing at least one parameter in accordance with which the electromagnetic stimulation is applied;
(b) ceasing to apply the electromagnetic stimulation; and
(c) maintaining stimulation parameters in accordance with which the electromagnetic stimulation is applied.

38. The method of claim 37, further comprising:
identifying at least one type of neglect disorder to which the patient is prone; and
based at least in part on the identified type of neglect disorder, selecting a target neural population to which the electromagnetic stimulation is directed.

39. The method of claim 37, further comprising:
identifying at least one type of neglect disorder to which the patient is prone; and
based at least in part on the identified type of neglect disorder, selecting the neglect test administered to the patient.

40. The method of claim 37, further comprising identifying at least one type of neglect disorder to which the patient is prone, wherein the neglect disorder includes unilateral neglect.

41. The method of claim 37, further comprising identifying at least one type of neglect disorder to which the patient is prone, wherein the neglect disorder includes at least one of sensory neglect, motor neglect, representational neglect, personal neglect, and spatial neglect

42. The method of claim 37 wherein administering a neglect test includes administering a cognitive test that results in a cognitive response and a motor response by the patient, and wherein the method further comprises distinguishing the cognitive response from the motor response.

43. The method of claim 37 wherein applying electromagnetic stimulation to the patient includes applying electromagnetic stimulation to the patient's central nervous system.

44. The method of claim 37 wherein applying electromagnetic stimulation to the patient includes applying electromagnetic stimulation to the cortex of the patient's brain.

45. The method of claim 37, further comprising implanting at least one electrode within the patient's skull, and wherein applying electromagnetic stimulation includes applying electrical signals to the patient's brain via the at least one electrode.

46. The method of claim 45 wherein implanting at least one electrode includes implanting at least one electrode at least proximate to the patient's cortex.

47. The method of claim 45 wherein implanting at least one electrode includes implanting at least one electrode beneath the patient's cortex.

48. The method of claim 37 wherein applying electromagnetic stimulation to the patient includes applying electromagnetic stimulation via transcranial magnetic stimulation.

49. The method of claim 37 wherein applying electromagnetic stimulation to the patient includes applying electromagnetic stimulation transcranial direct current stimulation.

50. The method of claim 37 wherein applying electromagnetic stimulation includes applying electromagnetic stimulation to the parietal lobe.

51. The method of claim 37 wherein applying electromagnetic stimulation includes applying electromagnetic stimulation to improve the patient's verbal conceptualization.

52. The method of claim 37 wherein applying electromagnetic stimulation includes applying electromagnetic stimulation to effectuate a lasting reduction in the effect of the neglect disorder.

53. The method of claim 37 wherein applying electromagnetic stimulation includes applying electromagnetic stimulation to the parietal lobe, and wherein the method further comprises selecting a portion of the parietal lobe to which stimulation is applied based at least in part on the type of neglect the patient suffers from.

54. The method of claim 37 wherein changing at least one parameter in accordance with which the electromagnetic stimulation is applied includes changing at least one of a current, voltage and waveform of a stimulation signal applied to the patient.

55. The method of claim 37 wherein changing at least one parameter in accordance with which the electromagnetic stimulation is applied includes changing a location at which the stimulation signal is applied.

56. A method for treating a patient, comprising:
applying electromagnetic stimulation to provide a long-lasting improvement in neuropsychological functioning of the patient;
after applying the electrical stimulation, evaluating the functioning of the patient; and
based at least in part on results of the evaluation, performing at least one of the following functions:
(a) changing at least one parameter in accordance with which the electromagnetic stimulation is applied;
(b) ceasing to apply the electromagnetic stimulation; and
(c) maintaining stimulation parameters in accordance with which the electromagnetic stimulation is applied.

57. The method of claim 56 wherein evaluating the functioning of the patient includes administering a test to the patient.

58. The method of claim 56, further comprising:
selecting at least one type of cognitive functioning; and
based at least in part on the selected type of cognitive functioning, selecting a cognitive test, and wherein evaluating the functioning includes administering the cognitive test.

59. The method of claim 56 wherein applying electrical stimulation includes applying electrical stimulation to a patient having a neglect disorder.

60. The method of claim 56 wherein applying electromagnetic stimulation to the patient includes applying electromagnetic stimulation to the cortex of the patient's brain.

61. The method of claim 56, further comprising implanting at least one electrode within the patient's skull, and wherein applying electromagnetic stimulation includes applying electrical signals to the patient's brain via the at least one electrode.
